(19) United States
(12) Patent Application Publication
(10) Pub. No.: US 2005/0113662 A1
(43) Pub. Date: May 26, 2005

(54) Magnetic sensor and method for analysing a fluid

(75) Inventors: Nasr-Eddine Djennati, Manchester (GB); Emanuel Cohen, Manchester (GB)

Correspondence Address: QUARLES & BRADY STREICH LANG, LLP, ONE SOUTH CHURCH AVENUE, SUITE 1700, TUCSON, AZ, 85701-1621, US

(73) Assignee: Nasr-Eddine Djennati Emanuel Cohen

(21) Appl. No.: 10/475,829

(22) PCT Filed: Apr. 29, 2002
(86) PCT No.: PCT/GB02/01953

(30) Foreign Application Priority Data
Apr. 27, 2001 (GB) 0110313.4

(51) Int. Cl. A61B 5/05

(52) U.S. Cl. 600/407; 436/149

(57) ABSTRACT

An apparatus and method for analysing a fluid including means for generating a non-uniform magnetic field in a space and means for measuring the strength of the magnetic field arranged to enable a change in the field strength to be measured when a sample of fluid to be analysed is introduced into the space.

### STATEMENT OF RELATED APPLICATIONS

This application claims priority to Patent Cooperation Treaty Application No. PCT/GB 02/01953, filed on Apr. 29, 2002, and to United Kingdom Patent Application No. 0110313.4, filed on Apr. 27, 2001.

### BACKGROUND OF THE INVENTION

1. Field of the Invention

The present invention relates to an apparatus and method for analysing a fluid, particularly, although not exclusively, body fluids such as blood, to determine the presence and concentration of various substances in blood.

2. Description of the Related Art

Analysis of blood is widely practised in the medical treatment and diagnosis of humans and animals. A plurality of methods are known for analysing blood. Embodiments of the present invention seek to provide an alternative method for analysing blood.

Conventionally it is necessary for a sample of blood to be removed from a living body for analysis outside the body. This can be unpleasant and inconvenient, especially if frequent analysis of blood is required such as can be the case for a sufferer of diabetes where frequent analysis of the concentration of glucose in their blood is necessary. Embodiments of the present invention seek to provide an apparatus and method for non-invasive analysis of blood in the body.

### SUMMARY OF THE INVENTION

According to a first aspect of the present invention there is provided apparatus for analysing a fluid comprising means for generating a non-uniform magnetic field in a space, the field varying with linear distance in the space, and means for measuring the strength of the field over a linear distance to enable a change in the strength of the field to be measured when a sample of fluid to be analysed is introduced into the space.

According to a second aspect of the present invention there is provided a method for analysing fluid comprising the steps of: applying a non-uniform magnetic field which varies with linear distance in space to a sample of fluid to be analysed and measuring the field strength over a linear distance to determine a change in the strength of the field caused by the presence of the sample.

All materials become magnetized to some extent when placed in a magnetic field. The extent and sign of magnetisation depends upon the nature of the material. When a material becomes magnetised in a magnetic field it experiences a force. Where a fluid containing a number of materials which respond differently to a magnetic field is placed into a non-uniform magnetic field the different materials are subjected to different forces and will thus tend to migrate to different regions of the field. The different magnetic properties of the materials present in the fluid will influence the field in different ways. With a knowledge of the way in which different materials influence the field it is possible to infer their presence and also concentration in a fluid subjected to a non-uniform magnetic field, by measuring the way in which the presence of the fluid influences the magnetic field.

The means for generating the non-uniform magnetic field preferably comprises two opposed, spaced apart permanent magnets, such as rare earth magnets, arranged to generate a field between each other. The magnets may be mounted on a yoke, for example a soft iron yoke. The magnets are each preferably fitted with a shaped pole piece operative to introduce non-uniformity into the field.

The means for measuring the strength of the field may comprise a linear array of magnetic field detectors, such as Hall effect devices. Any Hall effect devices are preferably of high sensitivity, especially of sensitivity of at least 30 mVmAkG⁻¹. Any Hall effect devices preferably comprise a ternary material. The array preferably extends in a direction in which the field strength varies. Each detector in the array preferably produces an output that depends upon the strength of the magnetic field measured by the detector. The output from each detector is preferably transmitted to a processing means. The processing means, which is preferably an electric or electronic processing means and may comprise a computer, is preferably arranged to determine the change in magnetic field sensed by each detector on introduction of a sample to be analysed into the space.

One way in which this can be achieved is for the processing means to annul the output from each detector such that when the space is empty the output from each detector is zero. Then, on introduction of a sample into the space, the positive or negative output from each detector represents the change in field brought about by introduction of the sample.

Alternatively, the means for measuring strength of magnetic field may comprise a single field detector mounted on positional apparatus operative to move the detector to near the field over a linear distance.

The change in field strength along the direction in which the field is measured will be indicative of the constituents of a sample being analysed. The processing means is preferably arranged to compare the measured change in field strength with stored information and to provide an indication of the contents of a sample being analysed, based upon the comparison.

The processing means is preferably comprised in a control unit including a means for outputting information to a user, for example a display.

The apparatus is suitable for analysing blood whilst in the body. For this it is preferable that the means for generating a magnetic field and the means for detecting the magnetic field are adapted to be worn on the body. Conveniently, they may be comprised in a clip adapted to be worn on an ear lobe.

The apparatus and method enable rapid analysis of fluids, particularly blood, which may be analysed non-invasively.

Various other purposes and advantages of the invention will become clear from its description in the specification that follows and from the novel features particularly pointed out in the appended claims. Therefore, to the accomplishment of the objectives described above, this invention consists of the features hereinafter illustrated in the drawings, fully described in the detailed description of the preferred embodiment and particularly pointed out in the claims. However, such drawings and description disclose but one of the various ways in which the invention may be practiced.

### BRIEF DESCRIPTION OF THE DRAWINGS

FIG. 1 shows part of apparatus according to the invention.

FIG. 2 is a schematic block diagram of the magnetic system of FIG. 1.

FIG. 3 is a perspective view of the magnet system of the apparatus of FIG. 1.

FIG. 4 is a graph of magnetic field strength against distance measured for a sample of blood using the apparatus of FIGS. 1 to 3.

### DETAILED DESCRIPTION OF THE PREFERRED EMBODIMENTS

In order that the invention may be more clearly understood, an embodiment thereof will now be described by way of example with reference to the accompanying drawings.

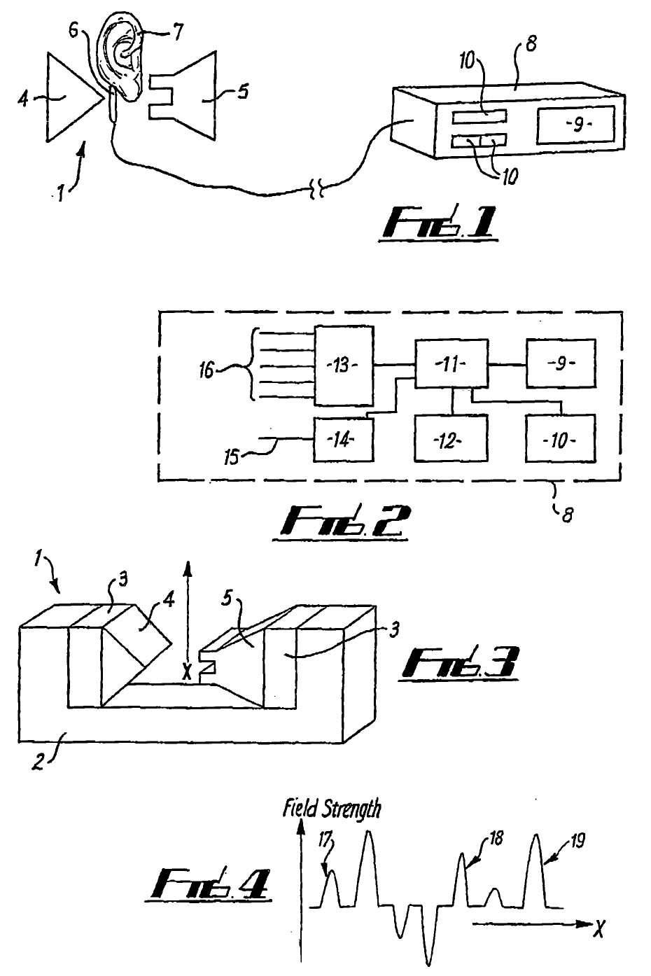

Referring to FIGS. 1 to 3, the apparatus comprises a magnet system 1. The magnet system comprises a generally square U-shaped soft iron yoke 2 comprising two spaced apart upstanding portions extending from a base portion. The upstanding portions define respective opposed, spaced apart, substantially parallel faces on each of which is mounted a rare earth permanent magnet 3. On each magnet 3 there is mounted a shaped soft iron pole piece 4,5 so that each magnet 3 is sandwiched between a face of the yoke 2 and a pole piece 4,5. Both pole pieces 4,5 substantially cover the face of the magnet 3 on which they are mounted.

One pole piece 4 is substantially triangular in cross-section and presents a wedge-shaped profile extending away from its magnet 3 and directed towards the other pole piece 5. The cross-sectional shape of the other pole piece 5 is that of a triangle with a flattened top from opposite sides of which extend rectangular protrusions defining a space therebetween. The magnets 3 are operative to generate a magnetic field in the space therebetween and the pole pieces 4,5 are operative to introduce non-uniformity into that field. In particular the pole pieces 4,5 introduce a variable field gradient extending in the direction indicated as X in FIG. 3.

The magnet system 1 further includes a linear array of Hall effect devices 6 extending between the magnets 3 in the X direction.

The magnet system 1 is disposed in the housing (not shown in FIG. 1, see FIG. 3) that enables it to be comfortably mounted on a person's ear 7 so that the person's ear lobe is disclosed in the region between the pole pieces 4,5.

The Hall devices of the array 6 are each electrically connected to a control unit 8. The control unit 8 comprises a housing having a display 9 and various user operable controls 10 on the outside and contains electronic circuitry 11,12 and 13 and an associated power supply 14. The circuitry comprises a multiplexer 13 connected to each of the Hall devices of the array 6 via connections 16, and to a microprocessor 11 to enable the Hall voltages of each Hall effect device to be measured in turn. The microprocessor 11 is also connected to the display 9, user operable controls 10, power supply 14 and a memory 12. The power supply 14 is also connected to each of the Hall devices of the array 6 via connection 15. The power supply 14 is operable both to supply power to the electronic circuitry comprising a control unit and a drive current to each of the Hall devices of the array 6. The microprocessor 11 is operative to monitor the Hall voltages measured from the Hall effect devices and to process this information to produce an output.

The processor is arranged such that when the magnet system 1 is disposed in the space the nominal Hall voltages measured for each of the Hall devices of the array 6 are all zero. Thus, upon introduction of a sample to be analysed, for example a person's ear lobe, into the space between the pole pieces 4,5 any Hall voltages then measured will represent the change in magnetic field strength measured by each Hall device brought about by the introduction of the sample.
materials become magnetised to some extent when placed in the magnetic field. Magnetisation (M) of the material is defined as magnetic moment per unit volume. It is relatively large and positive for ferromagnetic materials, relatively small and positive for paramagnetic materials and relatively small and negative for diamagnetic materials. In the presence of a magnetic field gradient these materials will experience a force per unit volume, F=M grad H where F is the force, M magnetisation and grad H field gradient. Thus, materials having a different magnetisation M will experience a different force. Some fluids, particularly blood, comprise a large number of materials of different magnetisation intimately mixed. The application of an intense non-uniform magnetic field to such a fluid will result in different forces being applied to the different materials leading to concentration gradients of those materials within the sample. The variation of concentration of materials within a sample in the region of a magnetic field leads to a variation in the magnetic field strength brought about by the presence of the various materials.

FIG. 4 is a graph showing the change in magnetic field strength along direction X of the apparatus of FIGS. 1 to 3 when a blood sample is introduced into the space between the pole pieces 4,5. The various peaks and troughs are indicative of the presence of various materials in the sample. For example the labelled peaks 17,18,19 of the graph indicate the presence of urea, glucose and creatine. It is possible to determine the presence and concentration of materials within the sample empirically, initially by comparing the results of analysis of a sample using the apparatus with those of a known technique, for example chemical analysis. Characteristic outputs representative of the presence of various materials can be determined and stored in the memory 12 of the control unit. The processor 11 is then operative to compare measured magnetic field distribution with stored information and thus to infer the presence and concentration of various materials in a sample being analysed. The change in magnetic field brought about by the presence of a particular material is proportional to the concentration of that material in the sample.

The apparatus is particularly suited to the non-invasive analysis of blood, but it will be apparent that it has many other applications too.

The above embodiment is described by way of example only. Many variations are possible without departing from the invention. Indeed, changes in the details, steps and components that have been described may be made by those skilled in the art within the principles and scope of the invention herein illustrated and defined in the appended claims. Therefore, while the present invention has been shown and described herein in what is believed to be the most practical and preferred embodiments, it is recognized that departures can be made therefrom within the scope of the invention, which is not to be limited to the details disclosed herein but is to be accorded the full scope of the claims so as to embrace any and all equivalent processes and products.

1. An apparatus for analysing a fluid, comprising:
means for generating a non-uniform magnetic field in a space, the field varying with linear distance in the space; and
means for measuring a magnetic field strength over a linear distance to enable a change in the magnetic field strength to be measured when a sample of fluid to be analysed is introduced into the space.

2. The apparatus as claimed in claim 1, wherein the means for generating the non-uniform magnetic field comprises two opposed, spaced apart, permanent magnets arranged to generate said non-uniform magnetic field between each other.

3. The apparatus as claimed in claim 1, wherein the means for generating the non-uniform magnetic field comprises two opposed, spaced apart, permanent magnets each mounted on a yoke and arranged to generate said non-uniform magnetic field between each other.

4. Apparatus as claimed in claim 1, wherein the means for generating the non-uniform magnetic field comprises two opposed, spaced apart, permanent magnets arranged to generate said non-uniform magnetic field between each other, wherein each permanent magnet is fitted with a shaped pole piece operative to introduce non-uniformity into the field.

5. The apparatus as clamed in claim 1, wherein the means for measuring the magnetic field strength comprises a linear array of magnetic field detectors.

6. The apparatus as claimed in claim 1, wherein the means for measuring the magnetic field strength comprises a linear array of magnetic field detectors extending in a direction in which the field strength varies.

7. The apparatus as claimed in claim 1, wherein the means for measuring the magnetic field strength comprises a single field detector mounted on a positional apparatus operative to move the field detector to scan a field over a linear distance in which the magnetic field strength varies.

8. The apparatus as claimed in claim 1, wherein the means for measuring the magnetic field strength comprises a linear array of magnetic field detectors and each of said detectors produces an output which depends upon the magnetic field strength measured by each detector and the output from each detector is transmitted to a processing means arranged to determine a change in the magnetic field strength sensed by each detector on introduction of a sample to be analysed into the space.

9. The apparatus as claimed in claim 1, wherein the means for measuring the magnetic field strength comprises a single field detector that produces an output which depends upon the magnetic field strength measured by the field detector, wherein the field detector is mounted on a positional apparatus operative to move the field detector to scan a field over a linear distance in which the magnetic field strength varies and the output from the field detector is transmitted to a processing means arranged to determine a change in magnetic field strength sensed by the field detector on introduction of a sample to be analysed into the space.

10. The apparatus as claimed in claim 1, wherein the means for measuring the magnetic field strength comprises a linear array of magnetic field detectors, wherein each of said detectors produces an output that depends upon the magnetic field strength measured by each detector, the output from each detector is transmitted to a processing means arranged to determine a change in magnetic field strength sensed by each detector on introduction of a sample to be analysed into the space, and the processing means is arranged to annul the output from each detector such that when the space is empty the output from each detector is zero and that on introduction of a sample into the space a positive or negative output from each detector represents the change in magnetic field strength brought about by introduction of the sample.

11. The apparatus as claimed in claim 1, wherein the means for measuring the magnetic field strength comprises a single field detector that produces an output which depends upon the magnetic field strength measured by the field detector, the field detector is mounted on a positional apparatus operative to move the field detector to scan a field over a linear distance, the output from the detector is transmitted to a processing means arranged to determine a change in magnetic field strength sensed by the field detector on introduction of a sample to be analysed into the space, and the processing means is arranged to annul the output from the field detector such that when the space is empty the output from each detector is zero and that on introduction of a sample into the space, a positive or negative output from each detector represents the change in magnetic field strength brought about by introduction of the sample.

12. The apparatus as claimed in claim 1 wherein, the means for measuring the magnetic strength comprises a linear array of magnetic field detectors, wherein each detector produces an output which depends upon the strength of the magnetic field measured by each detector and the output from each detector is transmitted to a processing means arranged to determine a change in magnetic field strength sensed by each detector on introduction of a sample to be analysed into the space, and wherein the processing means is arranged to compare the measured change in magnetic field strength with stored information and to provide an indication of contents of a sample being analysed based upon said comparison.

13. The apparatus as claimed in claim 1, wherein the means for measuring comprises a single field detector that produces an output which depends upon the magnetic field strength measured by the field detector, the field detector is mounted on a positional apparatus operative to move the field detector to scan a field over a linear distance, and the output from the detector is transmitted to a processing means arranged to determine a change in magnetic field strength sensed by the field detector on introduction of a sample to be analysed into the space, and wherein the processing means is arranged to compare the change in magnetic field strength with stored information and to provide an indication of contents of a sample being analysed based upon said comparison.

14. The apparatus as claimed in claim 1, wherein the means for measuring the magnetic field strength comprises a linear array of magnetic field detectors, wherein each detector produces an output which depends upon the strength of the magnetic field strength measured by each detector and the output from each detector is transmitted to a processing means arranged to determine the change in magnetic field strength sensed by each detector on introduction of a sample to be analysed into the space, and wherein the processing means further comprises a control unit including a means for outputting information to a user.

15. The apparatus as claimed in claim 1, wherein the means for measuring the magnetic field strength comprises a single field detector that produces an output which depends upon the magnetic field strength measured by the field detector, the field detector is mounted on a positional apparatus operative to move the field detector to scan a field over a linear distance, and the output from the field detector is transmitted to a processing means arranged to determine a change in magnetic field sensed by the field detector on introduction of a sample to be analysed into the space, and wherein the processing means further comprises a control unit including a means for outputting information to a user.

16. The apparatus as claimed in claim 1, wherein the means for generating a non-uniform magnetic field and the means for detecting the magnetic field strength are adapted to be worn on a human or animal body.

17. A method for analysing fluid comprising the steps of:
applying a non-uniform magnetic field that varies with linear distance in space to a sample of fluid to be analysed; and
measuring a magnetic field strength over a linear distance to determine a change in the magnetic field strength caused by the sample of fluid.

18. The method as claimed in claim 17, wherein the magnetic field strength measured is compared with stored information to provide an indication of the contents of the sample of fluid being analysed.

19. The method as claimed in claim 17, wherein the sample of fluid is blood.
